(19) United States
(12) Patent Application Publication
(10) Pub. No.: US 2025/0237663 A1
(43) Pub. Date: Jul. 24, 2025

(54) HIGH THROUGHPUT ASSAYS TO STUDY NUCLEOSOME REMODELING ENZYMES

(71) Applicant: EPICYPHER, INC., Durham, NC (US)

(72) Inventors: Martis W. Cowles, Chapel Hill, NC (US); Michael-Christopher Keogh, Cambridge, MA (US); Lu Sun, Apex, NC (US); Zu-Wen Sun, Brentwood, TN (US)

(21) Appl. No.: 18/698,542

(22) PCT Filed: Oct. 6, 2022
(86) PCT No.: PCT/US2022/077662

§ 371 Date: Apr. 4, 2024

Related U.S. Application Data
(60) Provisional application No. 63/252,740, filed on Oct. 6, 2021.

(51) Int. Cl. G01N 33/68; C12Q 1/37; G01N 1/30; G01N 21/64; G01N 33/543

(52) U.S. Cl. CPC G01N 33/6875; C12Q 1/37; G01N 1/30; G01N 21/6428; G01N 33/54313; G01N 2021/6439; G01N 2333/922; G01N 2333/96441

(57) ABSTRACT

Disclosed herein are improved methods to assay and quantify enzymatic activity of remodeling enzymes and identifying modulators of remodeling enzyme activity. Methods for performing a nucleosome remodeling assay can comprise steps of contacting a recombinant nucleosome substrate with a remodeling enzyme; binding the recombinant nucleosome substrate to a solid support; contacting the recombinant nucleosome substrate with a cleaving enzyme that recognizes a cleaving enzyme site of the recombinant nucleosome; activating the cleaving enzyme to cleave an exposed cleaving enzyme site on the recombinant nucleosome substrate to produce a cleaved nucleosome fragment; separating the cleaved nucleosome fragment from the solid support; and quantifying an amount of DNA associated with the cleaved nucleosome fragment, wherein the amount of DNA is associated with the nucleosome remodeling activity and/or detecting histones in the cleaved nucleosome fragment.

### STATEMENT OF PRIORITY

This application claims the benefit of U.S. Provisional Application Ser. No. 63/252,740, filed Oct. 6, 2021, the entire contents of which are incorporated by reference herein.

### FIELD OF INVENTION

The invention relates to assays for detecting and/or quantitating nucleosome remodeling activity using recombinant nucleosomes. The invention further relates to methods of using the assay to quantify enzymatic activity of remodeling enzymes and identifying modulators of remodeling enzyme activity.

### STATEMENT REGARDING ELECTRONIC FILING OF A SEQUENCE LISTING

A Sequence Listing in XML format, entitled 1426-40WO_ST26.xml, 8,192 bytes in size, generated on Oct. 5, 2022 and filed herewith, is hereby incorporated by reference in its entirety for its disclosures.

### BACKGROUND

Nucleosomes are the fundamental and repeating unit of chromatin, comprising a core histone octamer wrapped by ˜147 bp DNA (MARGUERON, 2010). Chromatin is more than a means to package the genome: it also regulates diverse cellular functions, including gene expression, chromosome transmission at mitosis, and DNA damage repair (BROWN, 2012; LAHTZ, 2011; LUNYAK, 2008; REIK, 2007). A key regulatory facet of these processes is mediated by nucleosome remodeling complexes that are recruited to sites of DNA methylation and/or histone modification. ATP-dependent remodeling subunits assemble, reformat, slide, or eject nucleosomes (collectively termed ‘remodeling’) to alter DNA accessibility for the transcription, replication, and repair machineries. Defective nucleosome remodeling is associated with diverse human disorders including schizophrenia (MCCARTHY, 2014), cardiovascular disease (BEVILACQUA, 2014), intellectual disability (LOPEZ, 2015; MCCARTHY, 2014) and cancer (ESTELLER, 2008; KUMAR, 2016; SPERLAZZA, 2015). Therefore, nucleosome remodelers are compelling therapeutic targets (MAYES, 2014; WANG, 2014; WU, 2016).

Recombinant nucleosomes, representing the full histone octamer wrapped in DNA, are ideal biochemical substrates for chromatin-focused drug discovery assays as they provide users with a homogenous substrate population that can be synthetically modified for analysis via direct readout methods (ALLIS, 2012). Remodeling complexes make multivalent interactions with the DNA and histones in chromatin, which cannot be replicated with isolated histone, peptide, or oligonucleotide substrates (LANGST, 2015).

The development of high-throughput methods to monitor the activity of nucleosome remodeling enzymes using physiological and fully defined recombinant nucleosome substrates is a major technical hurdle in the field. First-generation high-throughput compatible assays were developed using a FRET-based approach, wherein a recombinant nucleosome substrate contains fluorophores positioned on the DNA and a histone to result in high FRET (fluorescence resonance energy transfer). Following nucleosome remodeling the fluorophore pair are separated resulting in a reduction of FRET signal. While these FRET-based (or TR-FRET-based) assays are high-throughput, the nucleosome substrates are challenging and costly to generate at scale (ZHOU, 2016). These assays also have a relatively small assay window, requiring large amounts of substrate and/or enzyme to generate reliable assay signal. Further, these assays are only suitable for remodeling enzymes that prefer terminally positioned nucleosomes (i.e., histone octamer is positioned near the end of the DNA), limiting access to enzymes (e.g., ISWI family nucleosome remodeling enzymes) that prefer centrally positioned nucleosomes (i.e., histone octamer is positioned in center of DNA fragment). There is a need in the field therefore, to develop improved high-throughput nucleosome remodeling assays that exhibit improved assay performance metrics (e.g., signal to noise) and reduced assay costs.

### INVENTION SUMMARY

The present invention is based, in part, on the development of nucleosome remodeling assays that significantly improve signal to noise compared to current state of the art assays, thus reducing the amount of enzyme and substrates required to perform the assay. In one embodiment, a method for performing a nucleosome remodeling assay is provided comprising the steps of contacting a recombinant nucleosome substrate comprising a histone octamer, a nucleosomal DNA, a binding moiety and a cleaving enzyme site that is shielded by the histone octamer, with a remodeling enzyme; binding the recombinant nucleosome substrate to a solid support; contacting the recombinant nucleosome substrate with a cleaving enzyme that recognizes the cleaving enzyme site of the recombinant nucleosome; activating the cleaving enzyme to cleave an exposed cleaving enzyme site on the recombinant nucleosome substrate to produce a cleaved nucleosome fragment; separating the cleaved nucleosome fragment from the solid support; and quantifying an amount of DNA associated with the cleaved nucleosome fragment, wherein the amount of DNA is associated with the nucleosome remodeling activity and/or detecting histones in the cleaved nucleosome fragment. In an aspect, the remodeling enzyme is a remodeling enzyme complex.

In one example embodiment, the methods use recombinant nucleosome substrates comprised of a recombinant histone octamer wrapped in synthetic DNA containing a restriction enzyme site that is shielded from enzyme digestion by the histone octamer. Upon nucleosome remodeling, the restriction enzyme site is exposed. The nucleosomes can be bound to a solid support before or after remodeling, and are then cleaved by a restriction enzyme where the cleaving enzyme site, i.e., restriction enzyme site, is present. The cleaved nucleosome fragment can optionally be treated with proteinase K to remove the histone proteins. In one example, the DNA is quantified using a DNA intercalating dye (such as PicoGreen™). The quantification of DNA is proportional to the amount of nucleosome remodeling activity. These assays can be used for biomedical research and characterization of compounds that affect nucleosome remodeling activity.

The methods can further comprise contacting the recombinant nucleosome substrate and remodeling enzyme with a modulating agent prior to binding the recombinant nucleosome substrate.

In an aspect, the binding moiety is associated with the nucleosomal DNA, the histone octamer, or both. The binding moiety may be associated with the nucleosomal DNA at the 5′ end or at the 3′ end.

The methods may include one or more steps of washing the recombinant nucleosome substrate bound to the substrate.

In an aspect, contacting the recombinant nucleosome substrate with a cleaving enzyme is performed prior to or after binding the recombinant nucleosome substrate to the solid support.

In an embodiment, the recombinant nucleosome substrate comprises one or more histone modification and/or DNA modification.

In one embodiment, the cleaving enzyme is a DNA cleaving enzyme. The cleaving enzyme can be, without limitation, a restriction enzyme, nicking enzyme, or transposase. In one embodiment, the cleavage enzyme site is a restriction enzyme site. In an aspect, the restriction enzyme site is GATC. The restriction enzyme can be DpnII.

In an aspect, the solid support is a bead. The solid support can be a magnetic bead or a hydrophilic bead. In an aspect, the solid support is a surface. The solid surface can comprise the well of a plate. The solid support can comprise a binding partner of the binding moiety. The binding moiety and binding partner can be selected from, e.g., biotin and streptavidin, glutathione-S-transferase and glutathione, polyhistidine tag and immobilized metal affinity resin, FLAG and anti-FLAG antibody, and digoxigenin and a digoxigenin-specific antibody. One or more steps of the reaction may be performed in a microwell.

In an embodiment, separating the cleaved nucleosomes can comprise isolating a supernatant comprising the cleaved nucleosome fragment.

Methods can comprise quantifying the amount of DNA associated with the cleaved nucleosome fragment by contacting the cleaved nucleosome fragment with a nucleic acid stain. In an aspect, the nucleic acid stain can be an intercalating dye or a minor groove binder. Quantifying the DNA can be performed by, e.g., spectrophotometry, and detecting the histones can be performed by, e.g., immunoblot, ELISA, or mass spectrometry.

In an embodiment, the methods can further comprise deproteinating the cleaved nucleosome fragment. Deproteinating can be performed by contacting the cleaved nucleosome fragment with an enzyme such as proteinase K. Deproteinating and DNA staining may be performed in separate steps or at the same time.

Methods of identifying a compound that alters nucleosome remodeling are also provided, and can comprise contacting recombinant nucleosome substrate and a remodeling enzyme with a compound, performing the methods detailed herein, and quantifying an amount of DNA associated with the cleaved nucleosome fragment, wherein the amount of DNA is associated with the nucleosome remodeling activity, wherein a change in the quantity of DNA in the presence and absence of the modulating agent indicates that the compound alters nucleosome remodeling, and/or detecting histones in the cleaved nucleosome fragment, wherein a change in the type and/or quantity of histones in the presence and absence of the modulating agent indicates that the compound alters nucleosome remodeling.

Methods for performing a nucleosome remodeling assay with a library of recombinant nucleosome substrates are also provided. In an aspect, two or more of the recombinant nucleosome substrates in the library comprise a different pattern of one or more histone modification and/or DNA modification.

Compositions and kits for performing the methods of nucleosome remodeling assays are also provided.

These and other aspects of the invention are set forth in more detail in the description of the invention below.

### BRIEF DESCRIPTION OF THE DRAWINGS

FIG. 1. An example assay workflow using a 384-well plate. Compatible nucleosomes have restriction digestion motifs covered and shielded by the histone octamer at the starting position. After remodeling by ATP-dependent chromatin remodelers in 384-well assay plates, reactions are immobilized to magnet beads or assay plates through binding moieties on DNA. The mixture is then quenched and washed repeatedly before the bead bound nucleosomes are incubated with a compatible restriction enzyme. Only remodeled nucleosomes are released through restriction enzyme cleavage of DNA into supernatant. The supernatant is then separated from the solid support, digested by proteinase K, and measured for fluorescence from dsDNA intercalating dyes.

FIG. 2. Bead selection and incubation optimization. (Panel A) Head-to-head testing of commercially available streptavidin magnetic beads using a remodeling assay containing a SMARCA5 enzyme and recombinant nucleosome substrate. Following treatment with DPNII restriction enzyme, samples were then incubated with PicoGreen™ detection reagent with or without thermolabile proteinase K and read on a compatible microplate reader. Final data was denoted as the ratios of PicoGreen™ fluorescence signals from ATP+ samples over ATP-samples (-control samples). The reactions were read again after 1 hour. (Panel B) A timecourse experiment, wherein nucleosome—bead mixtures were incubated at ambient temperature for indicated time periods, before the unbound supernatant was separated from the beads, mixed with PicoGreen™ detection reagent, and quantified on a compatible microplate reader.

FIG. 3. Free DNA with or without the restriction enzyme motif (GATC) was subjected to the complete remodeling reaction, bound to a solid support, washed, treated with a restriction enzyme (DPNII), and then detected for quantitative fluorescence using PicoGreen™ stain. The final signal was plotted as relative fluorescence units (RFU) and analyzed by linear regression. As expected, DNA was only detected in samples that contain a restriction enzyme site, demonstrating the remarkable specificity of DPNII to only cleave at GATC sites.

FIG. 4. The nucleosome remodeling assay monitors ACF and SMARCA2/4/5 remodeling activity. Indicated concentrations of drosophila ACF complex, human SMARCA2/BRM, SMARCA4/BRG1 and SMARCA5/SNF2H were each mixed with recombinant nucleosome substrates and ATP and samples where then quenched at different time points. Linear regressions of early time points were plotted to reflect the kinetic linear range. As expected, an increase in remodeling activity was observed as the amount of enzyme and time increase.

FIG. 5. Indicated concentrations of human SMARCA4/BRG1 were mixed with unmodified or H3K14 acetylated recombinant nucleosome substrates and ATP. Samples were then quenched at different time points, bound to solid support, washed, cleaved with DPNII, treated with a proteinase K and PicoGreen™ enzyme cocktail, and then read on a microplate reader to quantify the final fluorescent readout. Interestingly, an increase in activity of SMARCA4/BRG1 was not observed when using nucleosomes that contain H3K14ac (vs. unmodified), which has been shown in some experimental systems to increase the activity of related family members.

FIG. 6. Remodeling assay recapitulates remodeler substrate preferences. (Panel A) Indicated concentrations of human SMARCA2/BRM was mixed with terminally (6-N-66) or centrally (50-N-66) positioned recombinant nucleosome substrates (depicted by graphics in bottom left) and ATP, before being quenched at different time points with optimized quench solutions. Final restriction enzyme released solution was mixed with detection reagent and read by a compatible microplate reader for final fluorescence readout. Linear regressions of early time points were plotted to reflect the kinetic linear range and comparisons of the activities between the two nucleosomes. The slopes for the linear ranges are represented as initial rates as in (Panel B). Similar initial rates are plotted for other remodelers in (Panel C) for quantitative comparisons.

FIG. 7. Remodeling assay recapitulates nucleosome substrate preferences by various remodeler complexes. (Panel A) Indicated concentrations of human SMARCA5, hACF1 complex, and hWiCH complex were each mixed with terminal (6-N-66) or central (50-N-66) positioned nucleosome substrates and ATP, before being quenched at different time points with optimized quench solutions. Slopes for the linear ranges are represented as initial rates as in FIG. 6. (Panel B) Ratios of slopes of terminal over central nucleosomes were plotted for each remodeler at indicated concentrations to represent the substrate preferences.

FIG. 8. Validation of nucleosome remodeling assays for high-throughput screening of inhibitor compounds using Z′ analysis. (Panel A) Nucleosome remodeling assays were performed in varying concentrations of DMSO using drosophila ACF1 complex. (Panel B) Z prime analyses were performed on assays using various human remodeling enzymes, including dmACF1, SMARCA2, SMARCA4 and SMARCA5, with or without ATP addition (N=24).

FIG. 9. Validation of nucleosome remodeling assays for inhibitor dose studies. Nucleosome remodeling assays were performed using a range of concentrations of the tool compound BRM014, which has been previously shown to target SMARCA2 and SMARCA4 family enzymes (Papillon, et al., J. Med. Chem. 2018, 61, 22, 10155-10172; doi:10.1021/acs/jmedchem.8b01318; Jagani et al., doi:10.1101/812628). IC50 values were generated using a four-parameter variable slope fitting algorithm.

FIG. 10. Assay rationales and corresponding enzyme titrations using either a FRET-based nucleosome remodeling assay (Panel A) or novel restriction enzyme-based nucleosome assay (Panel B). The optimal working concentrations of enzyme in each assay was boxed, showing that enzyme consumption is significantly reduced using the restriction enzyme-based assay vs. current FRET-based assays. (Panel C) details in different aspects of assay performances, along with estimated reagents costs shown in (Panel D).

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide”, “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

The present invention relates to assay methods for biochemical characterization of i) nucleosome remodeling enzymes and ii) agents, for example, inhibitors, that modulate the activity of nucleosome remodeling enzymes. The invention is based at least in part on the development of methods that have improved the throughput and sensitivity of nucleosome remodeling assays that use cleavage enzyme-based recombinant nucleosome substrates. As described herein, Applicant has developed an ultra-sensitive and versatile remodeling assay that directly exploits altered accessibility of nucleosomal enzymatic digestion site(s) in a non-radioactive setting. The assay has unprecedented advantages in sensitivity and is significantly enzyme-saving compared to other commercialized remodeling assays. The assay supports end-point readout and is compatible with a variety of distinct nucleosome substrates. The assay also enables the incorporation of stringent wash steps before or after the remodeling reaction by immobilizing the nucleosome substrate onto a solid support. The incorporation of wash steps makes this assay uniquely superior in avoiding compound interference for high-throughput screening compatible drug screening purposes.

While remodeling assays using recombinant nucleosome-based assay substrates containing a restriction enzyme site that is shielded by the histone octamer are known, see, e.g., International Patent Publication No. WO2018213719, the previous restriction enzyme based nucleosomes are relatively low-throughput, typically using gel electrophoresis methods to monitor DNA cleavage.

The present invention can be performed entirely in microwell plates (e.g., 96-, 384-, 1536-well), providing the first automation compatible assays that use cleavage enzyme-based nucleosome assay substrates. Further, these assays are highly sensitive, requiring less substrate and/or enzyme compared to FRET-based assays known in the art. Thus, together with the reduced manufacturing costs of generated cleavage-enzyme-based nucleosome substrates (vs. FRET-based substrates), the present invention provides improved high-throughput compatible assay metrics at dramatically reduced assay costs vs. current FRET-based assay substrates.

### Nucleosome Remodeling Methods

In the present invention, nucleosome remodeling assays are performed using a functionalized recombinant nucleosome substrate, which contains a binding moiety (e.g., biotin) and a cleavage enzyme site that is shielded by the histone octamer. In some embodiments, compounds can be added to the assay that modulate activity of nucleosome remodeling binding and/or activity. Before or after a nucleosome remodeling reaction, the recombinant nucleosome substrate is bound to a solid support (and optionally washed). Next, the nucleosome substrate is treated with a cleavage enzyme, which cleaves the recombinant nucleosome substrates containing an exposed cleavage enzyme site, thereby releasing nucleosomes into the supernatant that were ‘remodeled’ during the reaction. Finally, the supernatant containing the cleaved nucleosome fragments is optionally treated with a proteinase to degrade this histone octamer and DNA is quantified as a readout of nucleosome remodeling. As nucleosomes are remodeled, the conformation of the DNA associated with the nucleosome is altered; chromatin remodeling regulates DNA access on chromatin, impacting gene expression and genome repair. In one aspect, as nucleosome remodeling increases access to a cleaving site of the DNA increases, thereby increasing cleavage. PTMs can change the conformation of the histones, for example, by changing the charge, thereby increasing or reducing affinity for DNA. By way of example, histone acetylation decreases the positive charge of the histone proteins, reducing their affinity for DNA and causing the chromatin to open. Alternatively, chromatin remodeling enzymes (and complexes) can contain binding domains that interact with histone PTMs and/or DNA modifications, which in turn can positively and/or negatively affect the activity of the complex. Modulating agents can affect the ability of an enzyme to remodel the nucleosome, increasing or decreasing the accessibility, and therefore the amount of cleavage, of the DNA. Alternatively modulating agents can also disrupt the binding of the enzyme to the nucleosome or alter the enzymatic activity of the remodeling enzyme.

Nucleosome remodeling assays can comprise the steps of contacting a recombinant nucleosome substrate comprising a histone octamer, a nucleosomal DNA, a binding moiety and a cleaving enzyme site that is shielded by the histone octamer, with a remodeling enzyme.

The methods comprise binding the recombinant nucleosome substrate to a solid support. Linking of the nucleosome substrate to the solid support can provide a streamlined method to separate remodeled from non-remodeled nucleosome substrates, as only the remodeled substrates will be cleaved, thereby releasing DNA into the supernatant. In some embodiments, the nucleosome is linked to the solid support before the nucleosome remodeling reaction. In some embodiments, the nucleosome is linked to the solid support after the nucleosome remodeling reaction.

Methods can include contacting the recombinant nucleosome substrate with a cleaving enzyme that recognizes the cleaving enzyme site of the recombinant nucleosome, which may comprise a restriction endonuclease as the cleaving enzyme with a cleaving enzyme site specific for the cleaving enzyme. In some embodiments, the method further comprises activating the cleaving enzyme to cleave an exposed cleaving enzyme site on the recombinant nucleosome substrate to produce a cleaved nucleosome fragment. Example methods of activating the cleaving enzyme include administration of a divalent cation, for example, Zn²⁺, Cd²⁺, Ni²⁺, Mn²⁺, Co²⁺, Fe²⁺ or Mg²⁺. In an aspect, activating may also comprise providing ATP and/or S-adenosyl methionine (SAM, also referred to as AdoMet). In an aspect, the ATP can be hydrolyzed or non-hydrolyzed.

The cleaved nucleosome fragment can be separated from the solid support and separating may comprise isolating a supernatant comprising the cleaved nucleosome fragment. In an aspect, the separating may include a washing step to capture the cleaved nucleosome fragments from the uncleaved nucleosomes.

The method comprises a step of quantifying. In an aspect, the quantifying is determining an amount of DNA associated with the cleaved nucleosome fragment, wherein the amount of DNA is associated with the nucleosome remodeling activity. Quantifying the DNA can comprise contacting the cleaved nucleosome fragment with a nucleic acid stain, as described herein. The methods may comprise quantifying DNA by methods such as spectrophotometry.

The method may comprise in addition to quantifying the amount of DNA, detecting histones (e.g., the amount and/or type of histones or histone modifications) in the cleaved nucleosome fragment. In an aspect, the method comprises detecting histones in the cleaved nucleosome fragment without quantifying the amount of DNA. Detecting the histones may comprise, e.g., immunoblotting, ELISA, or mass spectrometry. In an aspect, the detecting can comprise evaluating histone modifications from the administering of modulating agents. In an aspect, detecting histones by amount or type comprises detecting a change in the type and/or quantity of histones in the presence and absence of the modulating agent, which indicates that the compound alters nucleosome remodeling.

Methods may further comprise the step of deproteinating the cleaved nucleosome fragment. In one aspect, the deproteinating comprises contacting the cleaved nucleosome fragment with an enzyme, e.g., a proteinase, e.g., proteinase K. The deproteinating and the DNA staining steps can be performed at the same time or at different times. In one aspect, the supernatant containing the cleaved nucleosome fragments is optionally treated with a proteinase to degrade the histone octamer and DNA is quantified as a readout of nucleosome remodeling, for example, with imaging of stained DNA of the cleaved nucleosome fragments.

The methods may comprise contacting the recombinant nucleosome substrate and remodeling enzyme with a modulating agent. The contacting the substrate with a modulating agent may occur after contacting the recombinant nucleosome substrate with a remodeling enzyme. The contacting the substrate with a modulating agent may occur prior to binding the recombinant nucleosome substrate to a solid support.

The methods can comprise a step of washing the recombinant nucleosome substrate bound to the substrate. Sample washing can be useful to optimize subsequent cleavage and/or detection reactions as well as mitigate interference effects caused by compounds (e.g., small molecule inhibitors) that affect restriction enzyme activity and/or DNA detection. Indeed, homogeneous (i.e., no-wash) assays are often limited by compound interference, which can complicate interpretation of assay results. See, Coussens, et al., Compound-Mediated Assay Interferences in Homogeneous Proximity Assays, Assay Guidance Manual, 2020.

The methods can comprise contacting the recombinant nucleosome substrate with a cleaving enzyme prior to the step of binding the recombinant nucleosome substrate to the solid support. In an aspect, the contacting the recombinant nucleosome substrate with a cleaving enzyme occurs subsequent to the step of binding the recombinant nucleosome substrate to the solid support.

The methods can comprise one or more steps of the reaction performed in a microwell. In an aspect, the solid support is a microwell, and multiple reactions can be performed in a high throughput manner on a plate comprising a plurality of microwells.

### Recombinant Nucleosome Substrate

A recombinant nucleosome substrate may comprise a recombinant mononucleosome. The recombinant nucleosome substrate may comprise one or more histone modification and/or DNA modification, i.e., is functionalized. A recombinant nucleosome may comprise a protein octamer, containing two copies each of histones H2A, H2B, H3, and H4, and optionally, linker histone HI. Each of the histones in the nucleosome is independently fully synthetic, semisynthetic, or recombinant. Methods of producing histones synthetically, semi-synthetically, or recombinantly are well known in the art.

In an aspect, the histone can comprise one or more post-translational modification. The histone PTM may be any PTM for which measurement is desirable. In some embodiments, the histone PTM is, without limitation, N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid, or any combination thereof.

In one embodiment, the post translational modification is selected from one or a combination of modifications listed in Tables 1(a)-1(f) of International Patent Publication WO2019169263, specifically incorporated herein by reference.

The histone mutation may be any mutation known in the art or any mutation of interest. In some embodiments, the histone mutations are oncogenic mutations, e.g., mutations associated with one or more types of cancer. Known oncogenic histone mutations include, without limitation, H3K4M, H3K9M, H3K27M, H3G34R, H3G34V, H3G34W, H3K36M, or any combination thereof.

Several naturally occurring histone variants are known in the art and any one or more of them may be included in the nucleosome. Histone variants include, without limitation, H3.3, H2A.Bbd, H2A.Z.1, H2A.Z.2, H2A.X, mH2A1.1, mH2A1.2, mH2A2, TH2B, or any combination thereof.

The DNA post-transcriptional modification may be any modification for which measurement is desirable. Known post-transcriptional DNA modifications include, without limitation, 5-methylcytosine, 5-hydroxymethylcytosine, 5-formylcytosine, 5-carboxylcytosine, 3-methylcytosine, 5,6-dihydrouracil, 7-methylguanosine, xanthosine, and inosine. In some embodiments, the DNA post-transcriptional modification is 5-methylcytosine, 5-hydroxymethylcytosine, 5-formylcytosine, 5-carboxylcytosine, 3-methylcytosine, or any combination thereof.

The recombinant mononucleosome may comprise a mix of recombinant and/or synthetic histone octamers, one or more of which may comprise post-translational modifications (PTMs). In some embodiments, the recombinant nucleosomes are polynucleosomes comprising a plurality of octamers. In an aspect, the polynucleosome comprises less than 6, 5, 4, or 3 histone octamers. In some embodiments, each histone octamer comprises the same PTM(s), e.g., the nucleosomes are homogenous. In other embodiments each histone octamer comprises different PTM(s), e.g., the nucleosomes are heterogeneous. The recombinant mononucleosomes can be as described in International Patent Publication No. WO2018213719, incorporated herein by reference in its entirety.

One preferred aspect of the invention is the use of recombinant substrates, which can be manufactured to contain one (or more) physiological or disease-relevant histone and/or DNA modifications. Similarly, the recombinant nucleosomes can be engineered to comprise a DNA modification to facilitate cleaving enzyme.

### Binding Moiety

In an aspect, the binding moiety is associated with the nucleosomal DNA, the histone octamer, or both. The binding moiety may be associated with the nucleosomal DNA at the 5′ end or at the 3′ end. In some embodiments, the binding moiety is associated with a histone protein. In some embodiments, the binding moiety is associated with both the nucleosomal DNA and histone protein.

The nucleosome can be bound to the solid support using any type of binding moiety known in the art, such as biotin, GST, His, FLAG, and others.

Binding partners of the binding moieties can be disposed on, within, or otherwise associated with, the solid support. Example binding moieties and their binding partners include: biotin and streptavidin, glutathione-S-transferase and glutathione, polyhistidine tag and immobilized metal affinity resin, FLAG and anti-FLAG antibody, and digoxigenin and a digoxigenin-specific antibody.

### Cleaving Enzyme Site

The cleaving enzyme site is a nucleic acid site shielded by the histone octamer prior to remodeling of the nucleosome. In an aspect, the nucleic acid site comprises DNA. The cleaving enzyme site may be designed on a recombinant nucleosome or may be naturally present in the nucleosome. The enzyme cleavage site could be located anywhere on the DNA sequence that associates with the histone octamer.

The cleaving enzyme site can be tailored for the cleaving enzyme used in the assays. As an example, where utilizing a nicking enzyme, such as a CRISPR nickase, the cleaving enzyme site can be selected based on the CRISPR nickase nicking enzyme cleavage preferences, with a guide molecule designed so as to allow cleaving at a desired cleaving enzyme site.

In an example embodiment, the cleaving enzyme site is a restriction enzyme site. In a certain embodiment, the restriction enzyme site is GATC. In an embodiment, the restriction enzyme is DpnII.

In an embodiment, the recombinant nucleosome comprises a DNA sequence with two or more GATC restriction sites. An example DNA sequence comprising two GATC restriction sites comprises:

(SEQ ID NO: 1)
GAATTCATCAGAATCCCGGTGCCGAGGCCGATCAATTGATCGTAGACAGC
TCTAGCACCGCTTAAACGCACGTACGCGCTGTCCCCCGCGTTTTAACCGC
CAAGGGGATTACTCCCTAGTCTCCAGGCACGTGTCAGATATATACATCGA
TGATGATGGATAGATGGATGATGGATGGATGGATGATGATGGATGAATAG
ATGGATGGATGAAGCTT.

In an embodiment, the recombinant nucleosome comprises a DNA sequence with no cleavage enzyme sites, which can be used as negative controls.

### Remodeling Enzyme

Remodeling enzymes can comprise a remodeling enzyme, or a remodeling enzyme complex. The term remodeling enzyme or remodeling complex refers to enzymes that interact with chromatin, in particular the DNA and/or histones in the chromatin. The remodeling enzyme may be an ATP-dependent chromatin remodeling complex, including, but not limited to, the imitation switch (ISWI) family, CHD, IN080, SWI/SNF and ATRX. Subfamilies of the ISWI family can be used, including ISW1A, IS1b, ISW2, NURF, CHRAC, ACF, WICH, RoRC, RSF, CERF, and orthologs and homologs thereof. Accordingly, exemplary remodeling enzymes include SWI/SNF, RSC, ISW1a (ATP-dependent) NuRD family, BPTF bromodomain PHD finger TF (see, e.g. Zahid et al., “Opportunity Knocks for Uncovering New Function of an Understudied Nucleosome Remodeling Complex Member, the Bromodomain PHD Finger Transcription Factor, BPTF” Curr Opin Chem Biol. 2021 August; 63: 57-67; doi: 10.1016/j.cbpa.2021.02.003), PBAF, INO80, ATP-utilizing chromatin assembly and remodeling factor (ACF) complex, including drosophila ACF complex (dmACF), Human SMARCA2/BRM (SMARCA2), Human SMARCA4/BRG1 (SMARCA4), and human SMARCA5/SNF2H (SMARCA5).

### Solid Support

Solid support as used herein encompasses a bead or a solid surface. The bead or surface may, in some instances, be coated.

In an embodiment, the solid support is a bead. The bead can be a magnetic bead. The bead can be a hydrophilic bead, for example, a hydrogel bead. The solid support may be a surface of a microwell. The surface may comprise a hydrogel surface.

One aspect of the invention relates to methods that use a solid support to bind the nucleosome assay substrate before or after the remodeling reaction. This assay modification allows for the incorporation of (optional) wash steps prior to cleaving enzyme cleavage and DNA detection.

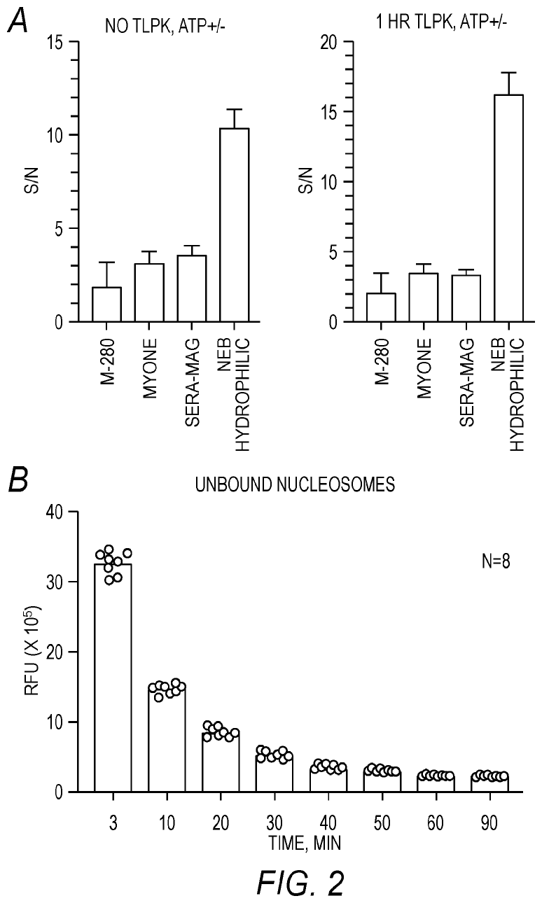

In some embodiments, the solid support is a bead (e.g., agarose or magnetic), which can be aqueous or bound to a surface (e.g., microwell plate). Applicant has systematically evaluated several commercially available beads, revealing that bead composition (e.g., hydrophobic vs. hydrophilic) can greatly impact assay sensitivity. In an exemplary embodiment, the solid support is a hydrophilic bead, which greatly improves assay signal compared to other types of commercially available beads (See, FIG. 2).

In an embodiment, the solid support or the nucleosome can comprise a nucleotide barcode sequence. Barcode identifier sequences are known in the art and typically comprise about 6 to 25 nucleotides in length. The barcode sequence and methods of incorporation and use can be as described in International Patent Publication No. WO 2019140082 and International Patent Publication WO2020132388, incorporated herein by reference. Barcoding can be used as needed to identify the cleaved nucleosomal DNA, for example by sample, individual, or other source identifying information.

### Cleaving Enzyme

In an aspect, the cleaving enzyme is a DNA cleaving enzyme. The invention can be performed using any type of enzyme that can cut and/or nick DNA at a specific sequence known in the art, including nickases and transposomes. For example, in some embodiments, the nucleosome contains a nickase site that is shielded by the histone octamer, which is specifically cleaved by a compatible nickase following nucleosome remodeling. Similar strategies can be deployed using transposomes. In the methods, the nucleosome substrate is treated with a cleaving enzyme, such as a restriction enzyme, which cleaves the recombinant nucleosome substrates containing an exposed restriction enzyme site, thereby releasing nucleosomes into the supernatant that were remodeled during the reaction. In an aspect, the cleaving enzyme is selected according to its preference for cleavage at terminal nucleosomes or central nucleosomes, as detailed further in the working examples.

### Restriction Enzyme

Restriction enzymes include Types I, II, II and IV enzymes. Restriction enzymes are commercially available and can be selected in one aspect, according to their recognition sequence. See, e.g., New England Biolabs, Tools and Resources, Selection Charts, Alphabetized List of Recognition Sequences. In an embodiment, the restriction enzyme is DpnII. In an embodiment, the restriction enzyme is DpnII and the remodeled nucleosome comprises DNA with one or more GATC restriction sites shielded by a histone prior to remodeling.

### Nicking Enzyme

In an example embodiment, a nicking enzyme can be used as the cleaving enzyme in the methods and assays described. Example nickases are endonucleases that hydrolyze one strand of a DNA duplex. Cleavage of one strand of a DNA duplex can be detected by use of probes, optionally detectable labeled, specific to the nicked site of the ssDNA Example restrictions enzyme nickases include Nt.BstNBI, Nt.CviPII, Nt.BspQI. N.Alw I, N.BvC IA and N.BvC D3. In an embodiment, the nickase is engineered from known or novel restriction enzymes. Hemiphophorothioate sites can be engineered as described in Walker, G. T. et al. (1992) Proc. Natl. Acad. Sci. USA 89, 392-396.

In an aspect, the nicking enzyme is a CRISPR nickase. CRISPR nickases are known in the art; for example SpCas9 mutant nickase comprising D10A mutation or H840A mutation for cleavage of a target strand or non-target strand, respectively, or AsCas12a nickase with a R1226A mutation. See Paul et al., “Mechanics of CRISPR-Cas12a and engineered variants on X-DNA” Nucleic Acids Research, Volume 50, Issue 9, 20 May 2022, pages 5208-522; doi: 10.1093/nar/gkabl272. Chen et al., Mol. Ther. VOLUME 8, p558-563; doi: 10.1016/j.omtn.2017.08.005. The nickase can be utilized with a guide molecule to conduct site specific nicking.

### Transposase

Transposases may be used with the methods of the present invention. Transposases include those comprising RNase H-like nuclease domains, such as Tn5, MuA, Mos1, Hermes, Serine and Tyrosine recombinases, including CTnDOT, Tn916, IS607 and TnpX, transposases comprising an HUH domain, including TnpA of IS91 or ISHp608, and helitron transposases, which can be as detailed in International Patent Publication WO2022056309, page 26, line 26-page 27, line 17, specifically incorporated by reference. See also nuclease guided transposase as described in WO2022150651 (DNA nuclease guided Transposases systems, Tn7-like transposition proteins with a Cas12k protein), WO2022147321 (Type I-B CRISPR Associated Transposase systems), WO2022076830 (Type I CRISPR Associated transposase systems), WO2021257997 (CAST); Li, et al., Int. J. Mol. Sci. 2020, 21(21), 8329; doi: 10.3390/ijms21218329 (Tn5 transposase in applied genomic research).

### Nucleic Acid Stain

In some embodiments, the DNA can be detected by the addition of nucleic acid stains, such as intercalating dyes (e.g., ethidium bromide and propidium iodide, SYBR™ Gold, SYBR™ Green arid SYBR™ Green IJ, cyanine based dyes), minor groove binders (e.g., DAPI, Hoechst, TOTO-1, indoles, imidazoles, and PicoGreen™) and other stains (e.g., acridine orange, 7-AAD, hydroxystilbamidine (H22845), and LDS 751). Stains may be selected based on desired detection methods.

Methods for Identifying Agents that Alter Nucleosome Remodeling

In some embodiments, agents can be added to the assay that modulate activity of nucleosome remodeling binding and/or activity. Accordingly, methods of identifying an agent that alters nucleosome remodeling are also disclosed. Methods comprise contacting a recombinant nucleosome substrate comprising a histone octamer, a nucleosomal DNA, a binding moiety and a cleaving enzyme site that is shielded by the histone octamer, with a remodeling enzyme; contacting the recombinant nucleosome substrate and the remodeling enzyme with an agent; binding the recombinant nucleosome substrate to a solid support; contacting the recombinant nucleosome substrate with a cleaving enzyme that recognizes the cleaving enzyme site of the recombinant nucleosome substrate; activating the cleaving enzyme to cleave an exposed cleaving enzyme site on the recombinant nucleosome substrate to produce a cleaved nucleosome fragment; separating the cleaved nucleosome fragment from the solid support; and quantifying an amount of DNA associated with the cleaved nucleosome fragment, wherein the amount of DNA is associated with the nucleosome remodeling activity, wherein a change in the quantity of DNA in the presence and absence of the agent indicates that the agent alters nucleosome remodeling, and/or detecting histones in the cleaved nucleosome fragment, wherein a change in the type and/or quantity of histones in the presence and absence of the agent indicates that the agent alters nucleosome remodeling.

The assays may be utilized to determine modulating agents of remodeling enzymes or remodeling enzyme complexes. Example druggable targets for cancer treatment can be explored in the assays, and are described, for example, in Mio, et al., Reading Cancer: Chromatin Readers as Druggable Targets for Cancer Treatment, Cancers 2019, 11(1), 61; doi:10.3390/cancers11010061, incorporated herein by reference, with particular reference to Tables 1 and 2.

In an embodiment, the evaluation of modulating agents is investigated with one or more SWI/SNF family of remodeling complex enzymes. SWI/SNF family chromatin remodeling complexes are of particular interest for targeted cancer treatment (HELMING, 2014; HOHMANN, 2014; PULICE, 2016; ZINZALLA, 2016). Mutations in SWI/SNF complex subunits are found in 20% of all human cancers, approaching the frequency of the most commonly mutated tumor suppressor, p53 (26%). Cells containing SWI/SNF mutant complexes exhibit abnormal chromatin remodeling activity, resulting in atypical gene expression that drives tumor initiation (KADOCH, 2013; SHAIN, 2013). Furthermore, SWI/SNF-mutated cancers present currently unexploited vulnerabilities that make characterizing and inhibiting SWI/SNF chromatin remodeling activity especially appealing for drug development (KADOCH, 2013; VANGAMUDI, 2015; WU, 2016). In the mutated ‘residual complexes’ simultaneous inhibition of the ATPase components (SMARCA2/4) is sufficient to arrest growth and induce cancer cell death (HELMING, 2014), a phenomenon referred to as synthetic lethality. The synthetic lethal relationship between SWI/SNF complex subunits may be exploited to target cancer cells while preserving healthy tissue (PULICE, 2017). Studies also indicate that targeting SWI/SNF remodeling complexes may be useful to treat cancers associated with loss of function mutations in the NURD family of remodeling enzymes (MOHD-SARIP; TEEUWSSEN; BOT; DE HERDT et al., 2017). Thus, inhibitors of nucleosome repositioning by SWI/SNF family complexes present a promising opportunity to discover highly needed novel treatments for therapeutically challenging cancers.

### Modulating Agents

An agent, e.g., compound, can be added to the nucleosome remodeling reaction that may alter the binding and/or activity of the nucleosome remodeling enzyme, i.e., a modulating agent. In an aspect, the modulating agent is a polypeptide, a small molecule, or a gene modifying enzyme.

In an embodiment, methods of the present invention utilize one or more small molecules as a modulating agent. In an embodiment, commercially available libraries of small molecules are available for screening purposes. An example library is available from Cayman Chemical Company, which includes epigenetic modulating agents. In an embodiment, modulating agents can be screened that modulate the activity of methyltransferases, demethylases, Histone Acetyl Transferases (HATs) such as HAT1, P300/CBP family, GNAT family, MYST family TFIIIC90, TAG1, SRC1, ACTR, p160, CLOCK, Histone deacetylases (HDACs) such as HDAC1, HDAC2, HDAC3, HDAC4, HDAC5, HDAC6, HDAC7, HDAC8, HDAC9, HDAC10, HDAC11, and SIRT1-7, and acetylated lysine reader proteins.

Polypeptides, including enzymes, small peptides, and antibodies can be utilized as a modulating agent. A gene modifying enzyme can be utilized as a modulating agent. In an embodiment, the gene modifying enzyme is a gene editing system. Example gene editing systems include a CRISPR system, a zinc finger nuclease system, or a TALE system.

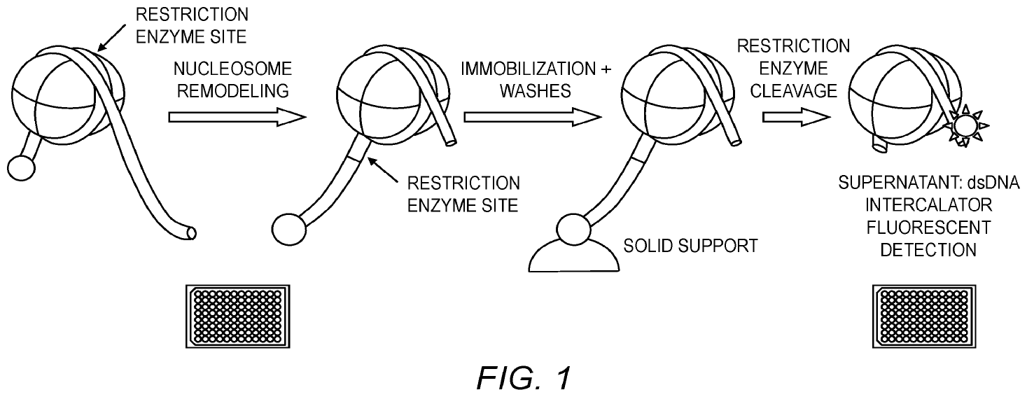

A CRISPR-Cas system can comprise a Class 1 or Class 2 CRISPR-Cas system, which may comprise a guide sequence engineered to specifically bind a polynucleotide of interest. The CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al., “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq. See, e.g., Malinin, et al., Nature Protocols, 16, 5592-5615 (2012). TALEN based gene editing is also contemplated and can be used in in vivo applications. See, S Becker, J Boch—Gene and Genome Editing, 2021. Zinc finger nuclease editing can also be utilized, and further modified to ensure high-precision gene editing. See, e.g., Conway et al., Molecular Therapy, 27:4, 10 Apr. 2019, Pages 866-877; Paschon et al. Nature Comm, 10.1133 (2019). Guide molecules can be engineered for site specific cleavage which may be used to design assays that target putative mutations. Gene editing tools are well known in the art, with advantages and comparison of the tools that can be considered for the desired application. Rahim et al., Int'l J. of Innovative Science and Research Tech., 6:8 (2021), incorporated herein by reference.

### Assays of Libraries of Nucleosome Remodeling

Methods for performing nucleosome library remodeling assays are also provided herein. The method can comprise contacting a library of recombinant nucleosome substrates, each recombinant nucleosome substrate comprising a histone octamer, a nucleosomal DNA, a binding moiety and a cleaving enzyme site that is shielded by the histone octamer, with a remodeling enzyme; binding each of the recombinant nucleosome substrates to a solid support; contacting the library of recombinant nucleosome substrates with a cleaving enzyme that recognizes the cleaving enzyme site of the recombinant nucleosome substrates; activating the cleaving enzyme to cleave one or more recombinant nucleosome substrate comprising an exposed cleaving enzyme site to produce one or more cleaved nucleosome fragments; separating the one or more cleaved nucleosome fragments from the solid support; and quantifying an amount of DNA associated with the one or more cleaved nucleosome fragments, wherein the amount of DNA is associated with the nucleosome remodeling activity and/or detecting histones in the one or more cleaved nucleosome fragments.

The methods can further comprise a step of washing each nucleosome bound to the substrate. In some embodiments, contacting each nucleosome substrate with a cleaving enzyme is performed prior to the step of binding the nucleosome substrate to the solid support. In some embodiments, contacting the nucleosome substrate with a cleaving enzyme is performed subsequent to the step of binding the nucleosome substrate to the solid support. The cleaving enzyme for each nucleosome of the library can be the same or different enzyme. One or more cleaving enzymes can be applied to the nucleosomal library.

In an aspect, two or more of the recombinant nucleosome substrates in the library comprise a different pattern of one or more histone modification and/or DNA modification.

Compositions and kits for use with the methods are also contemplated. Compositions comprising one or more recombinant nucleosome substrates comprising a binding moiety and a cleaving enzyme site that is shielded by a histone octamer; a cleaving enzyme; a DNA stain; a remodeling enzyme; and/or one or more solid supports in any combination are contemplated. The composition in one embodiment comprises the restriction enzyme DpnII, and the one or more recombinant nucleosomes comprise a cleaving site comprising GATC.

A further aspect of the invention relates to kits for use in the methods of the invention. Kits may comprise one or more recombinant nucleosome substrates comprising a binding moiety and a cleaving enzyme site that is shielded by a histone octamer; a cleaving enzyme; a DNA stain; a remodeling enzyme; one or more solid supports; and/or instructions for use, in any combination. The kit can further comprise modulating agents, carriers, buffers, containers, devices for administration of the components, and the like. The kit can further comprise labels and/or instructions for assay selection and execution. Such labeling and/or instructions can include, for example, information concerning the amount, and method of administration, detection and quantification for the assays detailed herein.

Having described the present invention, the same will be explained in greater detail in the following examples, which are included herein for illustration purposes only, and which are not intended to be limiting to the invention.

### Examples

### Example 1. Nucleosome Remodeling Assay

The present invention describes a method wherein an assay is performed using a nucleosome remodeling enzyme and recombinant nucleosome containing a binding moiety and a cleaving enzyme site that is shielded by the histone octamer. Optionally, a compound can be added to the nucleosome remodeling reaction that may directly or indirectly alter the binding and/or activity of the nucleosome remodeling enzyme. After the nucleosome remodeling reaction, the reactants can be bound to a solid support, optionally washed, and treated with a cleaving enzyme that cleaves “remodeled” nucleosomes (containing an exposed cleaving enzyme site) thereby releasing their nucleosomal DNA into the supernatant. Finally, the cleaved nucleosomal DNA is separated from the uncleaved nucleosomes, which can comprise a washing step, with the cleaved nucleosomal DNA then quantified. In a preferred embodiment, the cleaved nucleosomal fragments are separated from non-cleaved fragments by transferring the supernatant resulting from wash of the optional wash to a new tube or microwell plate (FIG. 1). In a preferred embodiment, deproteinization and DNA labeling of the cleaved nucleosomal fragments is performed in a single step using proteinase K and PicoGreen™, respectively.

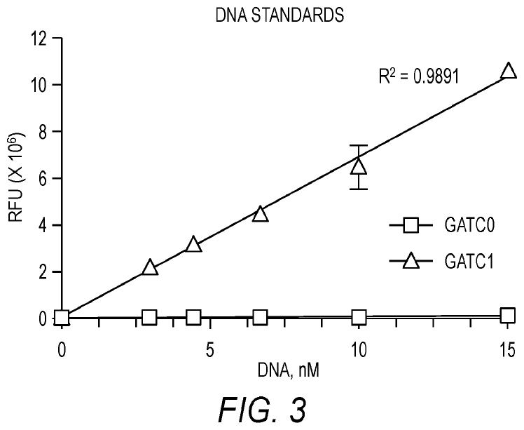

To begin to develop the novel remodeling assay, several key steps in the assay workflow were optimized. Multiple sources of streptavidin coated magnetic beads were explored to determine if bead composition influenced assay performance. Interestingly, it was found that NEB hydrophilic beads resulted in the best signal to background window (FIG. 2, panel A). Another key aspect of this invention is the direct detection of DNA using fluorescent dyes. Previous work has shown that proteins can affect DNA quantification using fluorescent dyes. It was found that removal of histones via proteinase digestion (Thermolabile proteinase K, TLPK) further improves assay signal under optimized conditions (FIG. 2, panel A right vs left). Finally, a timecourse experiment was performed to determine the optimal time needed for the nucleosomes to bind to the solid support, which were magnetic beads in this example. It was found found that a 1 hr (60 min) incubation is sufficient to saturate nucleosome-bead binding (FIG. 2, panel B). Finally, the specificity of a restriction enzyme to cleave at its target site was investigated. For these studies, free DNA (i.e., non nucleosome-bound DNA) was used that was engineered to contain 0 (GATC0) or 1 (GATC1) restriction enzyme site. In this example, the restriction enzyme DpnII was used, which is specific for GATC. Because the free DNA is not wrapped around the histone core, the restriction site is unshielded, making 100% of the substrate available for cleavage by the restriction enzyme. Nucleosome remodeling assays were performed as shown in FIG. 1 with free DNA substrates (GATC0 or GATC1) used in place of nucleosome substrates. As expected, a linear increase in assay signal was observed as more GATC1 substrate was added to the reaction (FIG. 3). Importantly, no recovery of GATC0 DNA was observed as this DNA lacks a restriction site and is thus not cleavable by the DpnII. These data show that the DpnII enzyme exhibits high target specificity in the context of the nucleosome remodeling assay workflow.

### Example 2. Chromatin Remodeling Enzymes Evaluation

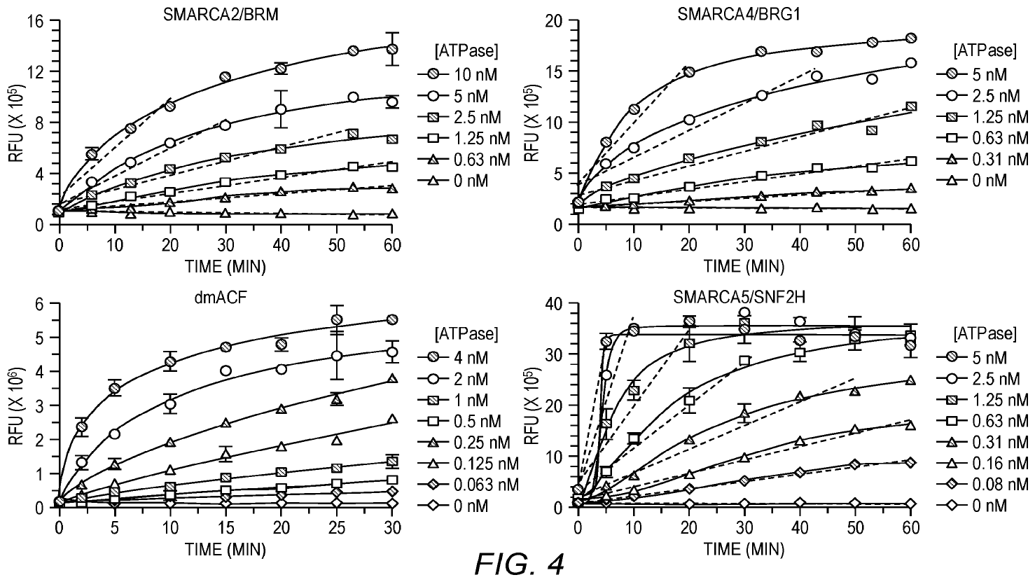

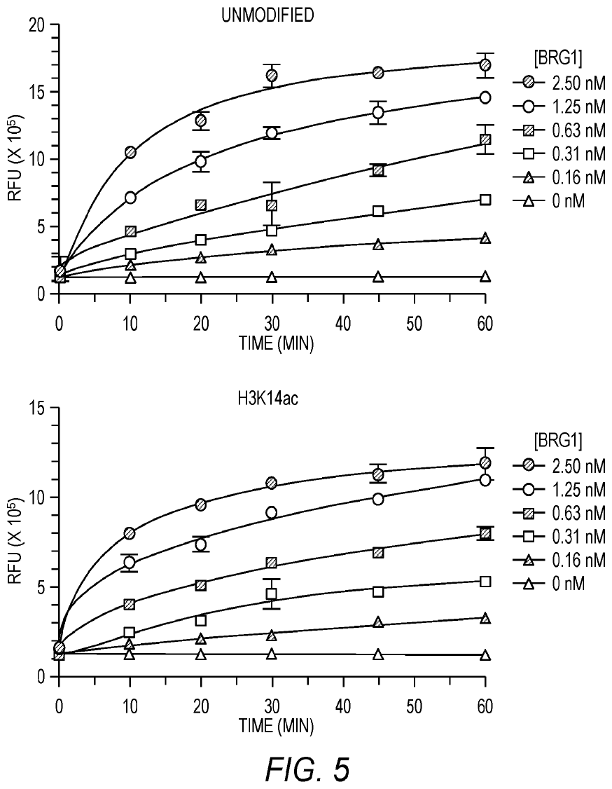

Here, the use of the novel nucleosome remodeling assays to monitor a set of different chromatin remodeling enzymes was validated. These included drosophila ACF complex (dmACF), human SMARCA2/BRM (SMARCA2), human SMARCA4/BRG1 (SMARCA4), and human SMARCA5/SNF2H (SMARCA5). For these studies, assays were performed using a range of enzyme concentrations (0-10 nM) and reactions were quenched after various amounts of time (0-60 min). Following restriction enzyme cleavage, released fragments were transferred to a new microwell plate, deproteinated using proteinase K, labeled using PicoGreen™ stain, and quantified using microwell plate reader. A time and enzyme dose dependent increase in assay signal was observed (FIG. 4). Similar results were also generated using BRG1 remodeling assays using unmodified or H3K14ac-modified recombinant nucleosome substrates. H3K14ac has been shown to increase the activity of remodeling enzymes in yeast but has yet to be tested using human enzyme. Interestingly, the results show that the activity of the human BRG1 enzyme is not increased in the presence of H3K14ac nucleosomes (vs unmodified) (FIG. 5). These experiments demonstrate how modified nucleosomes can be easily incorporated into biochemical studies using the present novel methods.

### Example 3. Nucleosome Positioning

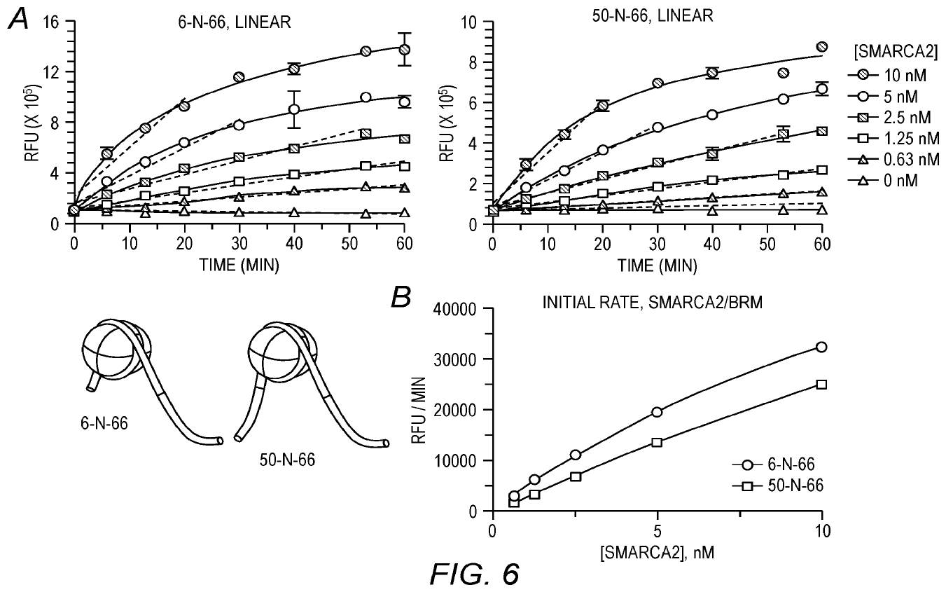

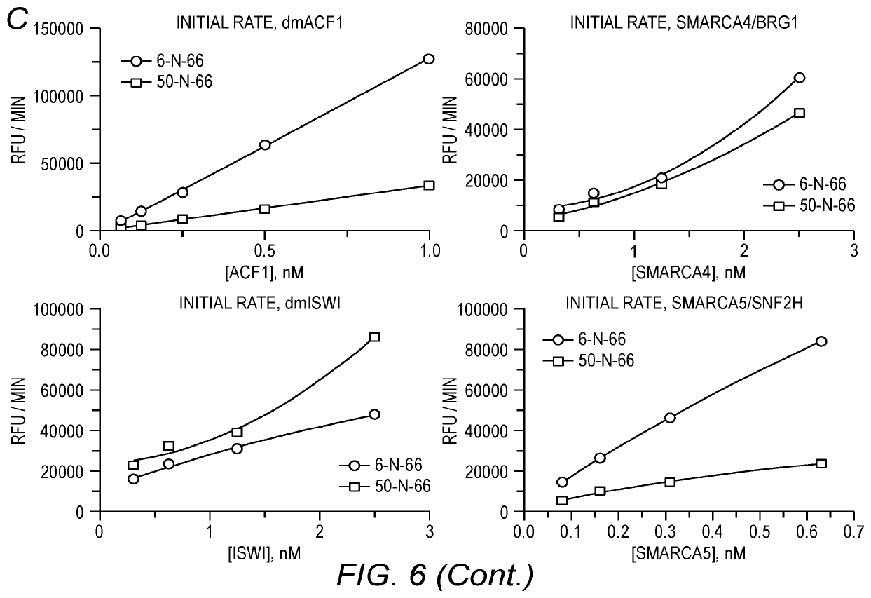

As some chromatin remodeling enzymes are involved in nucleosome spacing regulation and highly sensitive to the flanking DNA lengths of nucleosome substrates, It was then tested how the assay is adapted with nucleosome substrate species with differential flanking DNA states. Specifically, some remodelers are known to work better with terminally positioned nucleosomes, or nucleosomes with a longer flanking DNA on one side than the other. Meanwhile, other remodelers prefer centrally positioned nucleosomes, or nucleosomes with similar lengths of free DNA flanking both the entry and the exit side of the octamer core. The assay was performed with 6-N-66 (terminal) nucleosomes or 50-N-66 (central) nucleosomes when faced with various remodelers. See bottom left images in FIG. 6, panel A for a schematic of the nucleosomes used in this study. The result recapitulates such substrate preferences agreeing to previous publications. To be exact, it was found that SMARCA2 and SMARCA4 do not have a significant substrate preference (FIGS. 6, panels B-C), SMARCA5 and dmACF strongly prefer the terminally positioned nucleosomes (as indicated by increased assay signal), whereas dmISWI prefers to reposition centrally positioned nucleosomes (FIG. 6, panel C).

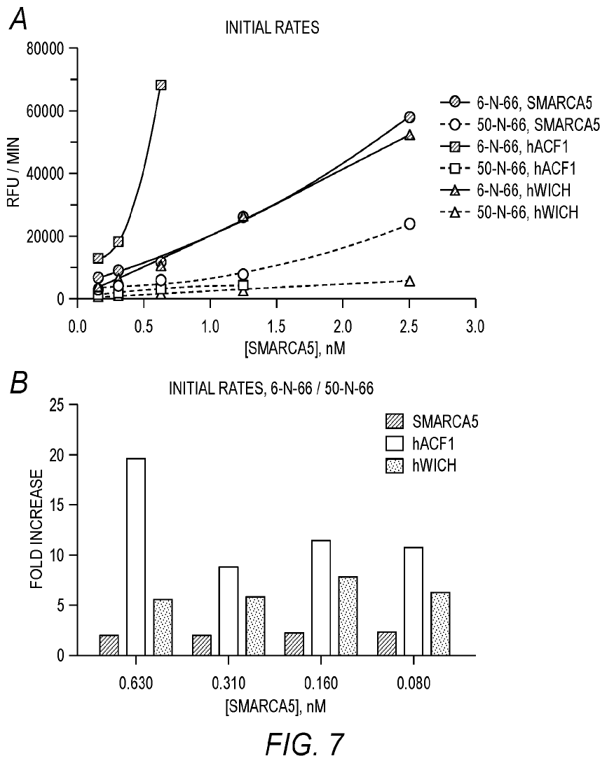

It was next asked if the assay could be used to resolve assay preference using enzyme complexes (vs single enzyme subunits in FIG. 6). For example, while the single ATPase SMARCA5 is capable of repositioning nucleosomes, in human cells it can be paired with various forms of accessory subunits that catalyze different reactions. Using this novel assay using either centrally or terminally positioned nucleosomes, it was found that when SMARCA5 is co-purified with the ACF1 subunit (into complex hACF1), it shows a drastic increase of activity with the terminal nucleosomes (FIG. 7, panel A). However, if SMARCA5 forms a complex with the WiCH subunit (into complex hWiCH), SMARCA5 complex shows similar reaction rate with terminal nucleosomes, but with a reduced activity with the centrally positioned nucleosomes (FIG. 7, panel A). In both cases, the complex forms of SMARCA5 show a stronger terminal to central bias in nucleosome selection as substrates (FIG. 7, panel B).

### Example 4. High Throughput Screening Assay

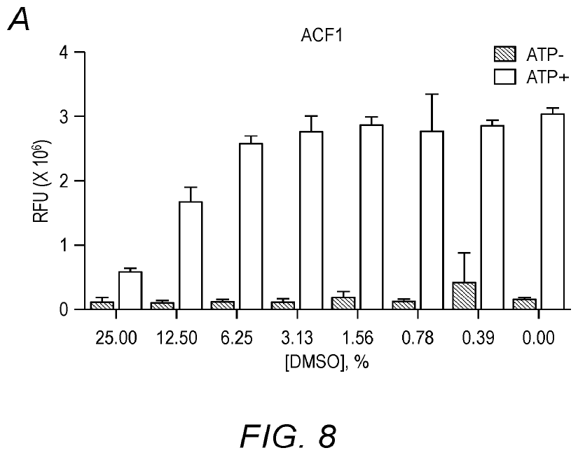

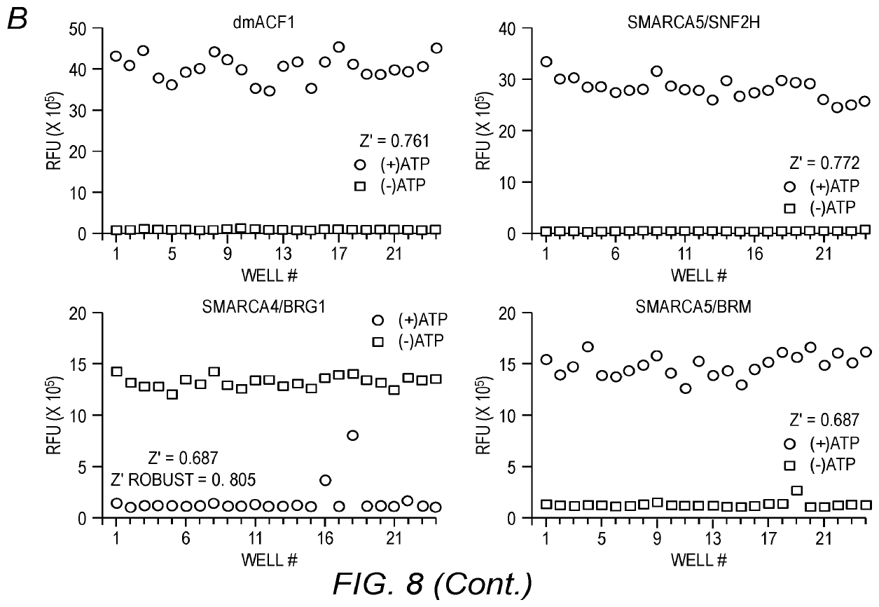

The present invention is compatible with high throughput screening applications. DMSO is the most widely used solvent for small molecule inhibitor compounds. To determine the potential impact of DMSO concentration, the assay was performed with serial dilutions of % DMSO in the assay buffer. It was found with the dmACF complex remodeling that the assay is tolerant to DMSO up to 6% without any noticeable pitfalls, which is well above the most commonly used DMSO (<1%) concentration in HTS assays (FIG. 8, panel A). The consistency of the assay was also tested by parallelizing the assay in multiple wells (N=24), with or without ATP The Z prime (Z′) value was then calculated for reactions using a set of remodeling enzymes, including dmACF1 (5 nM), SMARCA5 (10 nM), SMARCA4 (10 nM), and SMARCA2 (20 nM) (FIG. 8, panel B). Z′ value above 0.6 is generally considered HTS compatible in assay development. Notably, all enzymes were used at a quarter of the consumption as would be used in commercially available FRET-based nucleosome remodeling assays (EpiCypher; 16-4201).

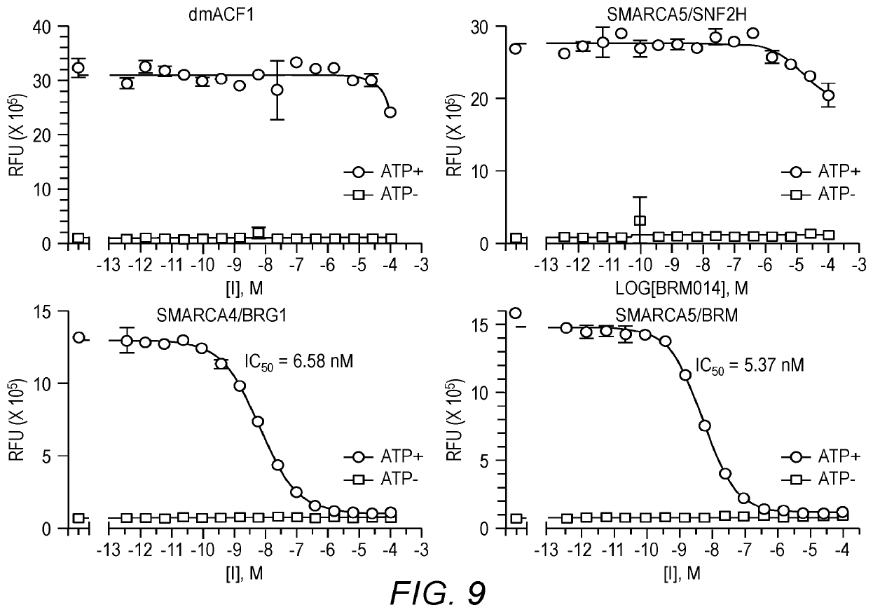

In addition to compound screening, the assay can also be used for inhibition dose response tests. These assays are typically used to confirm/further characterize putative hits from a primary screen. Using a published tool inhibitor BRM014 (Novartis) against various remodeling enzymes, its high specificity was recapitulated against SMARCA2 and SMARCA4, both with low nM IC50 values, but not against other remodelers such as dmACF or human SMARCA5 (FIG. 9).

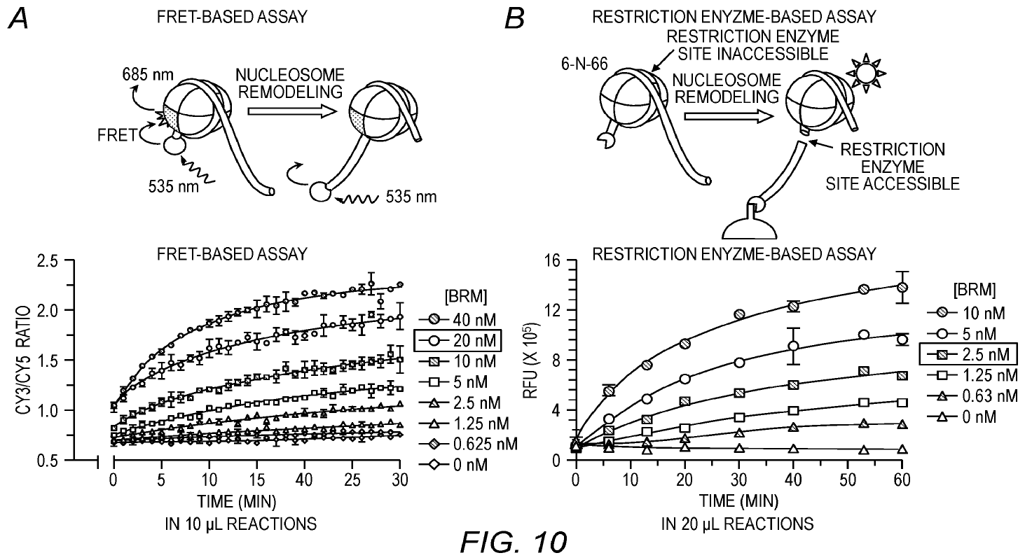

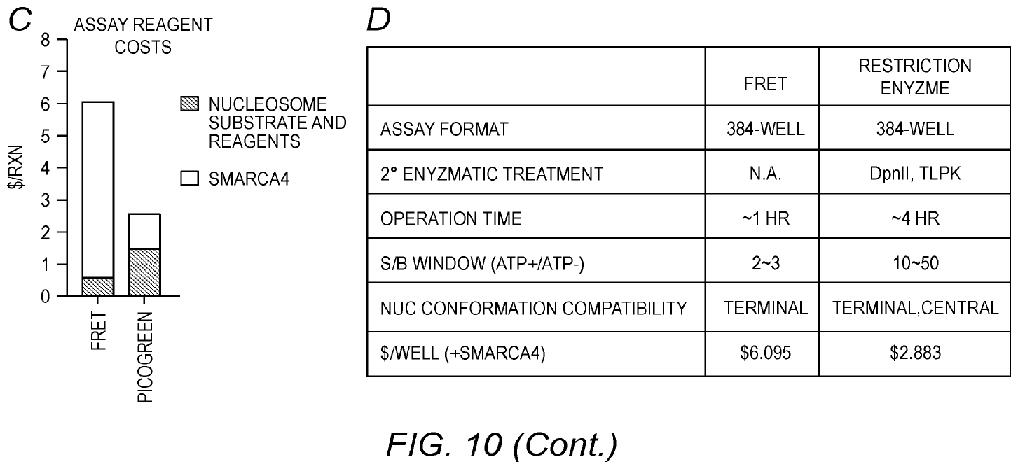

The present invention requires less enzyme compared to current FRET-based nucleosome remodeling assays (EpiCypher; 16-4201), which can dramatically reduce assay cost. To demonstrate the high sensitivity of the present assay, the restriction enzyme-based assay was compared with a FRET-based assay (FIG. 10, panels A-B). It was found that the restriction enzyme-base assay consumes as little as 25% of enzyme (vs FRET-based), which is the costliest and most difficult component to acquire for HTS remodeling assays (FIG. 10, panels A-B). While being more labor intense, the new assay is irreplaceably advantageous in the wide compatibility with different species of nucleosome substrates and minimal impact from compound interferences due to the thorough washes (FIG. 10, panels C-D).

### REFERENCES

ALLIS, C. D.; MUIR, T. W. Spreading chromatin into chemical biology. Chembiochem, 12, n. 2, p. 264-279, Jan. 24, 2011.
BEVILACQUA, A.; WILLIS, M. S.; BULTMAN, S. J. SWI/SNF chromatin-remodeling complexes in cardiovascular development and disease. Cardiovasc Pathol, 23, n. 2, p. 85-91, March-April 2014.
BROWN, S. J.; STOILOV, P.; XING, Y. Chromatin and epigenetic regulation of pre-mRNA processing. Hum Mol Genet, 21, n. R1, p. R90-96, Oct. 15, 2012.
CAREY, T. S.; CAO, Z.; CHOI, I.; GANGULY, A. et al. BRG1 Governs Nanog Transcription in Early Mouse Embryos and Embryonic Stem Cells via Antagonism of Histone H3 Lysine 9/14 Acetylation. Mol Cell Biol, 35, n. 24, p. 4158-4169, December 2015.
DANN, G. P.; LISZCZAK, G.; BAGERT, J. D.; MULLER, M. M. et al. ISWI chromatin remodellers sense nucleosome modifications to determine substrate preference. Nature, Aug. 2, 2017.
ESTELLER, M. Epigenetics in cancer. N Engl J Med, 358, n. 11, p. 1148-1159, Mar. 13, 2008.
FERREIRA, H.; FLAUS, A.; OWEN-HUGHES, T. Histone modifications influence the action of Snf2 family remodelling enzymes by different mechanisms. J Mol Biol, 374, n. 3, p. 563-579, Nov. 30, 2007.
FIERZ, B.; MUIR, T. W. Chromatin as an expansive canvas for chemical biology. Nat Chem Biol, 8, n. 5, p. 417-427, Apr. 17, 2012.
HELMING, K. C.; WANG, X.; ROBERTS, C. W. Vulnerabilities of mutant SWI/SNF complexes in cancer. Cancer Cell, 26, n. 3, p. 309-317, Sep. 8, 2014.
HOHMANN, A. F.; VAKOC, C. R. A rationale to target the SWI/SNF complex for cancer therapy. Trends Genet, 30, n. 8, p. 356-363, August 2014.
KADOCH, C.; HARGREAVES, D. C.; HODGES, C.; ELIAS, L. et al. Proteomic and bioinformatic analysis of mammalian SWI/SNF complexes identifies extensive roles in human malignancy. Nat Genet, 45, n. 6, p. 592-601, June 2013.
KUMAR, R.; LI, D. Q.; MULLER, S.; KNAPP, S. Epigenomic regulation of oncogenesis by chromatin remodeling. Oncogene, 35, n. 34, p. 4423-4436, Aug. 25, 2016.
LAHTZ, C.; PFEIFER, G. P. Epigenetic changes of DNA repair genes in cancer. J Mol Cell Biol, 3, n. 1, p. 51-58, February 2011.
LANGST, G.; MANELYTE, L. Chromatin Remodelers: From Function to Dysfunction. Genes (Basel), 6, n. 2, p. 299-324, Jun. 12, 2015.
LOPEZ, A. J.; WOOD, M. A. Role of nucleosome remodeling in neurodevelopmental and intellectual disability disorders. Front Behav Neurosci, 9, p. 100, 2015.
LUNYAK, V. V.; ROSENFELD, M. G. Epigenetic regulation of stem cell fate. Hum Mol Genet, 17, n. R1, p. R28-36, Apr. 15, 2008.
MARGUERON, R.; REINBERG, D. Chromatin structure and the inheritance of epigenetic information. Nat Rev Genet, 11, n. 4, p. 285-296, April 2010.
MAYES, K.; QIU, Z.; ALHAZMI, A.; LANDRY, J. W. ATP-dependent chromatin remodeling complexes as novel targets for cancer therapy. Adv Cancer Res, 121, p. 183-233, 2014.
MCCARTHY, S. E.; GILLIS, J.; KRAMER, M.; LIHM, J. et al. De novo mutations in schizophrenia implicate chromatin remodeling and support a genetic overlap with autism and intellectual disability. Mol Psychiatry, 19, n. 6, p. 652-658, June 2014.
MOHD-SARIP, A.; TEEUWSSEN, M.; BOT, A. G.; DE HERDT, M. J. et al. DOC1-Dependent Recruitment of NURD Reveals Antagonism with SWI/SNF during Epithelial-Mesenchymal Transition in Oral Cancer Cells. Cell Rep, 20, n. 1, p. 61-75, Jul. 5, 2017.
PETTY, E.; PILLUS, L. Balancing chromatin remodeling and histone modifications in transcription. Trends Genet, 29, n. 11, p. 621-629, November 2013.
PULICE, J. L.; KADOCH, C. Composition and Function of Mammalian SWI/SNF Chromatin Remodeling Complexes in Human Disease. Cold Spring Harb Symp Quant Biol, 81, p. 53-60, 2016.
REIK, W. Stability and flexibility of epigenetic gene regulation in mammalian development. Nature, 447, n. 7143, p. 425-432, May 24, 2007.
SHAIN, A. H.; POLLACK, J. R. The spectrum of SWI/SNF mutations, ubiquitous in human cancers. PLoS One, 8, n. 1, p. e55119, 2013.
SOSHNEV, A. A.; JOSEFOWICZ, S. Z.; ALLIS, C. D. Greater Than the Sum of Parts: Complexity of the Dynamic Epigenome. Mol Cell, 62, n. 5, p. 681-694, Jun. 2, 2016.
SPERLAZZA, J.; RAHMANI, M.; BECKTA, J.; AUST, M. et al. Depletion of the chromatin remodeler CHD4 sensitizes AML blasts to genotoxic agents and reduces tumor formation. Blood, 126, n. 12, p. 1462-1472, Sep. 17, 2015.
ST PIERRE, R.; KADOCH, C. Mammalian SWI/SNF complexes in cancer: emerging therapeutic opportunities. Curr Opin Genet Dev, 42, p. 56-67, February 2017.
STRAHL, B. D.; ALLIS, C. D. The language of covalent histone modifications. Nature, 403, n. 6765, p. 41-45, Jan. 6, 2000.
TURNER, B. M. Histone acetylation and an epigenetic code. Bioessays, 22, n. 9, p. 836-845, September 2000.
VANDEMARK, A. P.; KASTEN, M. M.; FERRIS, E.; HEROUX, A. et al. Autoregulation of the rsc4 tandem bromodomain by gcn5 acetylation. Mol Cell, 27, n. 5, p. 817-828, Sep. 7, 2007.
VANGAMUDI, B.; PAUL, T. A.; SHAH, P. K.; KOST-ALIMOVA, M. et al. The SMARCA2/4 ATPase Domain Surpasses the Bromodomain as a Drug Target in SWI/SNF-Mutant Cancers: Insights from cDNA Rescue and PFI-3 Inhibitor Studies. Cancer Res, 75, n. 18, p. 3865-3878, Sep. 15, 2015.
WANG, X.; HASWELL, J. R.; ROBERTS, C. W. Molecular pathways: SWI/SNF (BAF) complexes are frequently mutated in cancer-mechanisms and potential therapeutic insights. Clin Cancer Res, 20, n. 1, p. 21-27, Jan. 1, 2014.
WU, Q.; SHARMA, S.; CUI, H.; LEBLANC, S. E. et al. Targeting the chromatin remodeling enzyme BRG1 increases the efficacy of chemotherapy drugs in breast cancer cells. Oncotarget, 7, n. 19, p. 27158-27175, May 10, 2016.
ZHOU, C. Y.; NARLIKAR, G. J. Analysis ofNucleosome Sliding by ATP-Dependent Chromatin Remodeling Enzymes. Methods Enzymol, 573, p. 119-135, 2016.
ZINZALLA, G. A New Way Forward in Cancer Drug Discovery: Inhibiting the SWI/SNF Chromatin Remodelling Complex. Chembiochem, 17, n. 8, p. 677-682, Apr. 15, 2016.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for performing a nucleosome remodeling assay, the method comprising
a. contacting a recombinant nucleosome substrate comprising a histone octamer, a nucleosomal DNA, a binding moiety and a cleaving enzyme site that is shielded by the histone octamer, with a remodeling enzyme;
b. binding the recombinant nucleosome substrate to a solid support;
c. contacting the recombinant nucleosome substrate with a cleaving enzyme that recognizes the cleaving enzyme site of the recombinant nucleosome;
d. activating the cleaving enzyme to cleave an exposed cleaving enzyme site on the recombinant nucleosome substrate to produce a cleaved nucleosome fragment;
e. separating the cleaved nucleosome fragment from the solid support; and
f. quantifying an amount of DNA associated with the cleaved nucleosome fragment, wherein the amount of DNA is associated with the nucleosome remodeling activity and/or
g. detecting histones in the cleaved nucleosome fragment.

2. The method of claim 1, wherein the remodeling enzyme is a remodeling enzyme complex.

3. The method of claim 1, further comprising, prior to step b), contacting the recombinant nucleosome substrate and remodeling enzyme with a modulating agent.

4. The method of claim 1, wherein the binding moiety is associated with the nucleosomal DNA, the histone octamer, or both, optionally wherein the binding moiety is associated with the nucleosomal DNA at the 5′ end or at the 3′ end.

5. (canceled)

6. The method of claim 1, further comprising a step of washing the recombinant nucleosome substrate bound to the substrate.

7. The method of claim 1, wherein the step c) contacting the recombinant nucleosome substrate with a cleaving enzyme is performed prior to the step b) binding the recombinant nucleosome substrate to the solid support.

8. The method of claim 1, wherein the recombinant nucleosome substrate comprises one or more histone modification and/or DNA modification.

9. The method of claim 1, wherein the cleaving enzyme is a DNA cleaving enzyme, optionally wherein the cleaving enzyme is a restriction enzyme, nicking enzyme, or transposase.

10. (canceled)

11. The method of claim 1, wherein the cleavage enzyme site is a restriction enzyme site, optionally wherein the restriction enzyme site is GATC, optionally wherein the restriction enzyme is DpnII.

12-13. (canceled)

14. The method of claim 1, wherein the solid support is a bead, optionally a magnetic bead, optionally a hydrophilic bead, or a surface, optionally the well of a plate.

15-18. (canceled)

19. The method of claim 1, wherein separating comprises isolating a supernatant comprising the cleaved nucleosome fragment.

20. The method of claim 1, wherein the quantifying comprises contacting the cleaved nucleosome fragment with a nucleic acid stain, optionally wherein the nucleic acid stain is an intercalating dye or minor groove binder, or wherein the quantifying is by spectrophotometry.

21-22. (canceled)

23. The method of claim 1, wherein the detecting the histones is by immunoblot, ELISA, or mass spectrometry.

24. The method of claim 1, further comprising deproteinating the cleaved nucleosome fragment, optionally wherein the deproteinating comprises contacting the cleaved nucleosome fragment with proteinase K, optionally wherein the deproteinating and DNA staining are performed at the same time.

25-26. (canceled)

27. The method of claim 1, wherein the solid support comprises a binding partner of the binding moiety, optionally wherein the binding moiety and binding partner are selected from: biotin and streptavidin, glutathione-S-transferase and glutathione, polyhistidine tag and immobilized metal affinity resin, FLAG and anti-FLAG antibody, and digoxigenin and a digoxigenin-specific antibody.

28. (canceled)

29. The method of claim 1, wherein one or more steps of the reaction is performed in a microwell, optionally wherein the quantifying comprises using a microwell plate reader.

30. (canceled)

31. A method of identifying an agent that alters nucleosome remodeling, comprising:
a. contacting a recombinant nucleosome substrate comprising a histone octamer, a nucleosomal DNA, a binding moiety and a cleaving enzyme site that is shielded by the histone octamer, with a remodeling enzyme;
b. contacting the recombinant nucleosome substrate and the remodeling enzyme with an agent;
c. binding the recombinant nucleosome substrate to a solid support;
d. contacting the recombinant nucleosome substrate with a cleaving enzyme that recognizes the cleaving enzyme site of the recombinant nucleosome substrate;
e. activating the cleaving enzyme to cleave an exposed cleaving enzyme site on the recombinant nucleosome substrate to produce a cleaved nucleosome fragment;
f. separating the cleaved nucleosome fragment from the solid support; and
g. quantifying an amount of DNA associated with the cleaved nucleosome fragment, wherein the amount of DNA is associated with the nucleosome remodeling activity, wherein a change in the quantity of DNA in the presence and absence of the agent indicates that the compound alters nucleosome remodeling, and/or
h. detecting histones in the cleaved nucleosome fragment, wherein a change in the type and/or quantity of histones in the presence and absence of the agent indicates that the compound alters nucleosome remodeling.

32. (canceled)

33. A method for performing a nucleosome remodeling assay, the method comprising:
a. contacting a library of recombinant nucleosome substrates, each recombinant nucleosome substrate comprising a histone octamer, a nucleosomal DNA, a binding moiety and a cleaving enzyme site that is shielded by the histone octamer, with a remodeling enzyme;
b. binding each of the recombinant nucleosome substrates to a solid support;
c. contacting the library of recombinant nucleosome substrates with a cleaving enzyme that recognizes the cleaving enzyme site of the recombinant nucleosome substrates;
d. activating the cleaving enzyme to cleave one or more recombinant nucleosome substrate comprising an exposed cleaving enzyme site to produce one or more cleaved nucleosome fragments;
e. separating the one or more cleaved nucleosome fragments from the solid support; and
f. quantifying an amount of DNA associated with the one or more cleaved nucleosome fragments, wherein the amount of DNA is associated with the nucleosome remodeling activity and/or
g. detecting histones in the one or more cleaved nucleosome fragments.

34-36. (canceled)

37. A composition for carrying out the method of claim 1, comprising:
a. one or more recombinant nucleosome substrates comprising a binding moiety and a cleaving enzyme site that is shielded by a histone octamer; and one or more of
b. a cleaving enzyme;
c. a DNA stain;
d. a remodeling enzyme; and
e. one or more solid supports.

38. (canceled)

39. A kit for carrying out the method of claim 1, comprising
a. one or more recombinant nucleosome substrates comprising a binding moiety and a cleaving enzyme site that is shielded by a histone octamer; and one or more of
b. a cleaving enzyme;
c. a DNA stain;
d. a remodeling enzyme;
e. one or more solid supports; and
f. instructions for use.


SEQUENCE LISTING (1 sequence)

SEQ ID NO: 1 - DNA, 217 bp, synthetic construct
  source 1..217: mol_type = other DNA
gaattcatcagaatcccggtgccgaggccgatcaattgatcgtagacagctctagcaccgcttaaacgcacgtacgcgctgtcccccgcgttttaaccgccaaggggattactccctagtctccaggcacgtgtcagatatatacatcgatgatgatggatagatggatgatggatggatggatgatgatggatgaatagatggatggatgaagctt